(19) United States
(12) Patent Application Publication
(10) Pub. No.: US 2026/0248636 A1
(43) Pub. Date: Aug. 27, 2026

(54) URINE COLLECTION SYSTEMS HAVING ONE OR MORE OF VOLUME, PRESSURE, OR FLOW INDICATORS, AND RELATED METHODS

(71) Applicant: PureWick Corporation, Covington, GA (US)

(72) Inventors: Seth Schneider, Social Circle, GA (US); Claire Gloeckner, Lilburn, GA (US); Gregory Mann, Covington, GA (US); Damien Marechal, Claix (FR); Yolanda Rhodes, Cypress, TX (US)

(21) Appl. No.: 19/534,156

(22) Filed: Feb. 9, 2026

Related U.S. Application Data
(63) Continuation of application No. 17/808,354, filed on Jun. 23, 2022, now Pat. No. 12,575,960.
(60) Provisional application No. 63/214,570, filed on Jun. 24, 2021.

(51) Int. Cl. A61F 5/451; A61F 5/44

(52) U.S. Cl. CPC A61F 5/451; A61F 5/4404

(57) ABSTRACT

Examples relate to devices, systems, and methods for fluid collection such as urine. A urine collection system includes a urine collection device configured to be positioned at least proximate to a urethra of a user, a canister having an interior region, a first conduit in fluid communication with the urine collection device and the canister, and one or more of a urine volume indicator, a flow indicator, and/or a pressure indicator.

### CROSS-REFERENCE TO RELATED APPLICATIONS

This application is a continuation of U.S. application Ser. No. 17/808,354 filed Jun. 23, 2022, which claims priority to U.S. Provisional Patent Application No. 63/214,570 filed on Jun. 24, 2021, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to urine collection systems having volume, pressure, and/or flow indicators, and related methods. In an embodiment, a urine collection system includes a urine collection device configured to be positioned at least proximate to a urethra of a user, a canister having an interior region, a first conduit in fluid communication with the urine collection device and the canister, and one or more indicators configured to detect at least one property of the urine collection system. The one or more indicators may include at least a urine volume indicator secured to the canister and configured to detect a property relating at least to a volume of urine in the canister. The one or more indicators may include at least a flow indicator positioned to detect a property relating at least to a flow of fluid through the first conduit between the canister and the urine collection device. The one or more indicators may include at least a pressure indicator secured to the canister and configured to detect a property relating at least to a pressure in the interior region of the canister.

In an embodiment, a method of collecting urine from a user includes positioning a fluid collection device at least proximate to a urethra of a user, the fluid collection device being in fluid communication with an interior region of a canister through a first conduit. The method also includes activating a pump in fluid communication with the interior region of the canister through a second conduit effective to create a negative pressure in the canister and draw urine from the urine collection device through the first conduit into the interior region of the canister. The method also includes monitoring at least one property of the urine collection system with one or more indicators. The one or more properties may include at least a property relating at least to a volume of urine in the canister with a urine volume indicator secured to the canister. The one or more properties may include at least a property relating at least to a flow of fluid through the first conduit with a flow indicator in fluid communication with the first conduit and positioned between the canister and the urine collection device. The one or more properties may include at least a property relating at least to a pressure in the interior region of the canister with a pressure indicator secured to the canister.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a portable urine collection system, according to an embodiment.

FIG. 1B is a side view of a portable urine collection system, according to an embodiment.

FIG. 2A is a side view of a portable urine collection system, according to an embodiment.

FIG. 2B is a side view of a portable urine collection system, according to an embodiment.

FIG. 2C is a top isometric view of a base a portable urine collection system, according to an embodiment.

FIG. 2D is a side view of a portable urine collection system, according to an embodiment.

FIG. 2E is a side isometric view of a urine collection system in use, according to an embodiment.

FIG. 3 is a side view of a flow indicator for a urine collection system, according to an embodiment.

FIGS. 4A and 4B are front views of a pressure indicator of FIG. 1B positioned in a urine collection container.

FIG. 5 is a block diagram of a controller, according to an embodiment.

FIG. 6 is a flow diagram of a method of collecting fluid, according to an embodiment.

### DETAILED DESCRIPTION

Embodiments disclosed herein are related to urine collection systems having one or more of volume, pressure, or flow indicators, and related methods of using the same. The devices, systems, and methods disclosed herein are configured to collect fluids from an individual. The fluids collected by the fluid collection devices may include at least one of urine, vaginal discharge, penile discharge, reproductive fluids, blood, sweat, or other bodily fluids. At least one, some, or all embodiments disclosed herein include one or more indicators that provide a user and/or caregiver with quick information about the urine collection system. The indicators of at least one, some, or all embodiments described herein improve the urine collection system relative to other potential ways of assessing a condition of the urine collection system. For example, reading the volume of urine in the canister by sight must be done very close to the canister and may require glasses or corrective lenses for many users and/or caregivers. Moreover, in a conventional urine collection system, kinking of a conduit or overfilling of the canister may not be immediately evident without actual contact with the system. Technical improvements of urine collection systems described herein may include one or more of a urine volume indicator configured to detect a property relating at least to a volume of urine in a urine collection canister, a flow indicator configured to detect a property relating at least to a flow of fluid through the one or more portions of the system, or a pressure indicator configured to detect a property relating at least to a pressure in the interior region of the urine collection canister. One or more of these indicators in at least one, some, or all embodiments described herein provide low-cost ways for a user or caregiver to quickly see or assess a current condition of the system.

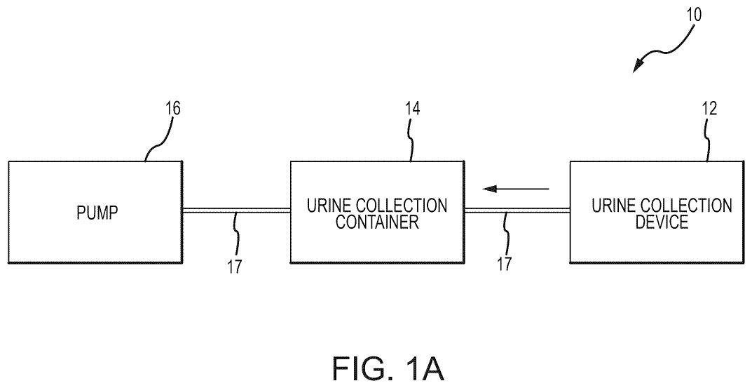

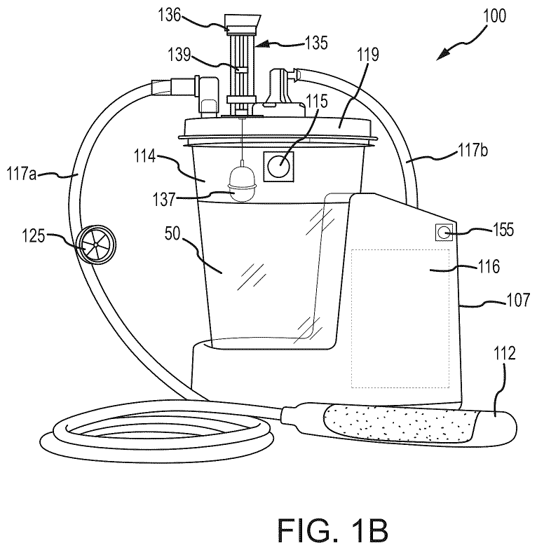

FIG. 1A is a block diagram of a fluid collection system 10, according to an embodiment. The fluid collection system 10 may be included in embodiments of fluid collection systems described herein. The system 10 includes a fluid collection device 12 (e.g., any of the fluid collection devices disclosed herein), a urine collection container 14, and a pump 16 (or vacuum source). The fluid collection device 12, the urine collection container 14, and the pump 16 may be fluidly coupled to each other via one or more conduits 17. For example, fluid collection device 12 may be operably coupled to one or more of the urine collection container 14 or the pump 16 via the conduit 17. In some embodiments, the pump 16 may be secured directly to the urine collection container 14. Fluid (e.g., urine or other bodily fluids) collected in the fluid collection device 12 may be removed from the fluid collection device 12 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the pump 16 either directly or indirectly. The suction force may be applied indirectly via the urine collection container 14. For example, the outlet of the conduit 17 may be disposed within or fluidly coupled to an interior region of the urine collection container 14 and an additional conduit 17 may extend from the urine collection container 14 to the pump 16. Accordingly, the pump 16 may apply suction to the fluid collection device 12 via the urine collection container 14. The suction force may be applied directly via the pump 16. For example, the outlet of the conduit 17 may be disposed within the pump 16. An additional conduit 17 may extend from the pump 16 to a point outside of the fluid collection device 12, such as to the urine collection container 14. In such examples, the pump 16 may be disposed between the fluid collection device 12 and the urine collection container 14.

The urine collection container 14 is sized and shaped to retain a fluid therein. The urine collection container 14 may include a bag (e.g., drainage bag), a bottle, a canister, or a cup (e.g., collection jar), or any other enclosed container for storing bodily fluid(s) such as urine. In some examples, the conduit 17 may extend from the fluid collection device 12 and attach to the urine collection container 14 at a first point therein. An additional conduit 17 may attach to the urine collection container 14 at a second point thereon and may extend and attach to the pump 16. Accordingly, a vacuum (e.g., suction) may be drawn through fluid collection device 12 via the urine collection container 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

FIG. 1B shows an example of a urine collection system 100, according to an embodiment. The urine collection system 100 may include a urine collection device 112, a first conduit 117a in fluid communication with an interior region of the urine collection device 112, a canister 114 having an interior region in fluid communication with the internal region of the urine collection device 112 via the first conduit 117a, a second conduit 117b in fluid communication with the interior region of the canister 114, and a pump 116 (housed in a base 107 or module) in fluid communication with the interior region of the canister 114 via the second conduit 117b. The urine collection device 112 may be configured to be positioned at least proximate to a urethra of a user. While the urine collection device 112 shown in FIG. 1B includes a female urine collection device, the urine collection device 112 may instead include a male urine collection device. PCT International Application No. PCT/US2019/029616, for example, describes various embodiments of both male and female fluid collection devices, the disclosure of which are incorporated in their entirety by this reference. Moreover, the urine collection device 112 may be interchangeable in the urine collection system 100 between different types, varieties, and sizes of male or female urine collection devices. Generally, the urine collection device 112 may include a surface sized to be positioned proximate or adjacent to the urethra and configured to wick urine or other fluids away from the user. Urine or other fluids may be wicked from the surface to a reservoir in the urine collection device 112.

The urine collection system 100 also includes the first conduit 117a in fluid communication with an interior region (e.g. reservoir) of the urine collection device 112 and an interior region of the canister 114. The first conduit 117a may be positioned between the urine collection device 112 and the canister 114. The urine collection system 100 also may include the second conduit 117b providing fluid communication between the pump 116 and the interior region of the canister 114. In some embodiments, the pump 116 may be secured directly to the canister 114, and the second conduit 117b may be absent from the urine collection system 100. The conduits 117a, 117b may include a flexible tube. In some embodiments, at least a portion of the first conduit 117a is substantially opaque, thereby inhibiting viewing of the urine within the first conduit 117a.

The canister 114 may be reusable and dishwasher safe, and may include a generally rigid material such as polycarbonate or glass. In some embodiments, the canister 114 may be disposable. The urine collection system 100 may be configured to accommodate different volumetric sizes of urine collection containers 114. In some embodiments, a lid 119 having multiple ports and/or adapters for attachment of the conduits 117a, 117b thereto may be secured or securable to the canister 114. Except for the ports and/or adapters for attachment of the conduits 117a, 117b, the canister 114 may be sealed and airtight such that the urine collected in the canister 114 does not leak or spill.

The pump 116 is in fluid communication with the interior region of the canister 114 and is configured to pull a vacuum (e.g., negative pressure) on the interior region of the canister 114 effective to draw the urine from the urine collection device 112 through the first conduit 117a into the canister 114. The pump 116 may be secured directly to the canister 114, or the conduit 117b may fluidly couple the pump 116 with the interior region of the urine collection container.

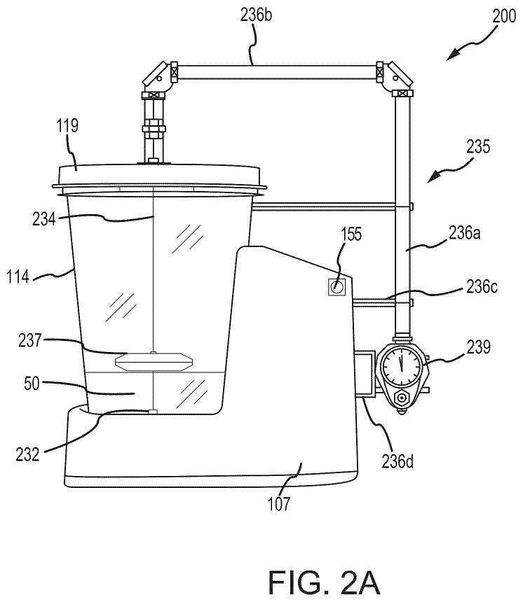

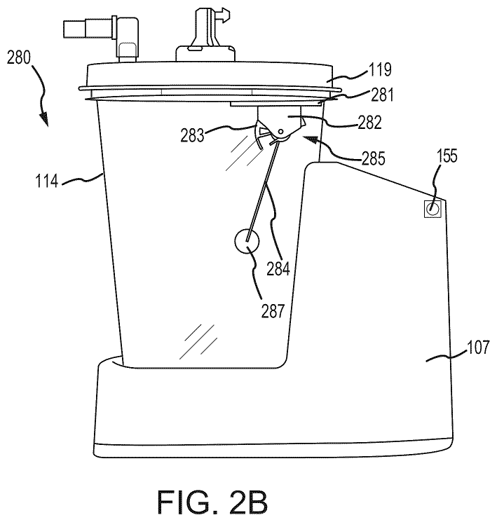

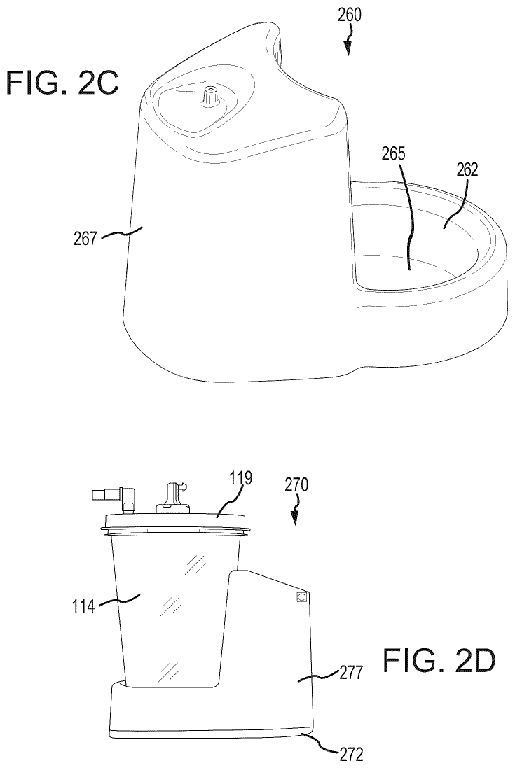

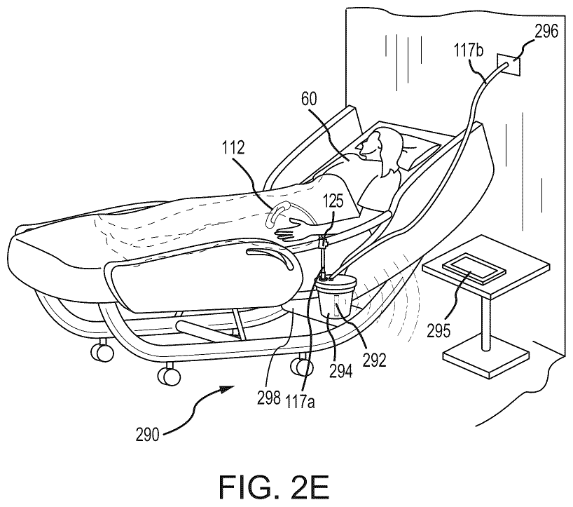

The pump 116 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 116 may provide a vacuum or suction to remove fluid from the fluid collection device 112. In some examples, the pump 116 may be powered by one or more batteries or other power sources. In some examples, the pump 116 may be sized and shaped to fit within a container support on a wheelchair. In some embodiments, the pump 116 may include a wall-mounted pump (shown in FIG. 2E). The pump 116 may include one or more of a switch, a button, a plug, a remote, or any other device suitable to activate the pump 116.

In some embodiments, the urine collection system 100 may include one or more (e.g., one, two, or three) of a urine volume indicator 135, a flow indicator 125, and/or a pressure indicator 115. Although the urine collection system 100 includes all three indicators of the urine volume indicator 135, the flow indicator 125, and the pressure indicator 115, some embodiments the urine collection system may include only one indicator or only any combination of two indicators of the urine volume indicator 135, the flow indicator 125, or the pressure indicator 115. In embodiments of the urine collection system 100 having two or more indicators of the urine volume indicator 135, the flow indicator 125, and the pressure indicator 115, the two or more indicators may be used simultaneously or one at a time.

With respect to the urine volume indicator 135, users and caregivers may desire to know the volume of fluid in the canister 114 and/or when the canister 114 needs to be emptied. In some embodiments, the urine volume indicator 135 is secured to the canister 114 and/or the lid 119 and is configured to detect a property relating at least to a volume of the urine 50 in the canister 114. For example, the urine volume indicator 135 may include a support member 136 secured or securable to at least one of the lid 119 or a sidewall of the canister 114. The urine volume indicator 135 may include a float member 137 configured to at least partially float on the urine 50 in the canister 114, thereby utilizing the buoyancy of the float member 137 on the urine 50 in the canister 114 to indicate or detect the property relating at least to the volume of the urine 50 in the canister 114. The float member 137 may include a hollow float member, such as a hollow ball.

In some embodiments, the urine volume indicator 135 may include one or more markers 139 configured to indicate at least a relative amount of the urine 50 in the canister 114. For example, the marker 139 may be movable on the support member 136 responsive to vertical movement of the float member 137 at least partially floating on the urine 50 in the canister 114. In some embodiments, the position of the marker 139 on the support member 136 indicates an approximate volume of the urine 50 in the canister 114. For example, when the canister 114 is substantially empty (e.g., holding substantially no urine 50), the marker 139 may be positioned at or near the bottom of the support member 136. When the canister 114 is substantially full of the urine 50, the marker 139 may be positioned at or near the top of the support member 136. In some embodiments, the support member 136 may include one or more markers indicating a percent capacity of the canister 114 being occupied by the urine 50. For example, the support member 136 may include a reference marker indicating 75% of the capacity of the canister 114 is occupied by the urine 50 when the marker 139 reaches the reference marker on the support member 136. In some embodiments, support member 136 includes one or more color reference markers. For example, the support member 136 may include (1) a green area indicating about 0% to about 50% of the capacity of the canister 114 is occupied by urine 50 when the marker 139 is positioned within the green area, (2) a yellow area indicating about 50% to about 75% of the capacity of the canister 114 is occupied by the urine 50 when the marker 139 is positioned within yellow area, and (3) a red area indicating the about 75% to about 100% of the capacity of the canister 114 is occupied by the urine 50 when the marker 139 is positioned within the red area.

In some embodiments, the urine collection system 100 includes an alert device 155 in communication with the urine volume indicator 135 and configured to produce an alert signal when the relative amount of the urine 50 in the canister 114 is at a predetermined volume. For example, the alert device 155 may be configured to produce the alert signal when the amount of the urine 50 in the canister 114 is approximately 75% or 90% of the capacity of the canister 114. The alert signal produced by the alert device 155 may include at least one of a visual (e.g., light) alert, an audible (e.g., sound) alert, or a haptic alert. In some embodiments, the urine volume indicator 135 is wired or wirelessly coupled to the pump 116 in the urine collection system 100 effective to activate (e.g., turn on) the pump 116 when the urine 50 in the canister 114 is below a predetermined volume and deactivate (e.g., turn off) the pump 116 when the urine 50 in the canister 114 is at or above a predetermined volume. The urine collection system 100 also may include a controller configured to facilitate communication between the urine volume indicator 135 and at least one (e.g., both) of the pump 116 and the urine alert device 155.

Turning ahead in the drawings, FIG. 2A is a side view of urine collection system 200 having a urine volume indicator 235, according to an embodiment. Unless otherwise noted, the urine collection system 200 can include any aspect of the urine collection system 100. For example, the urine collection system 200 may include one or more (e.g., all) of the pump 116, the canister 114, the lid 119, the conduits 117a, 117b (not shown), the urine collection device 112 (not shown), and/or the urine alert device 155.

The urine volume indicator 235 may be secured or securable to the canister 114 and configured to detect a property relating at least to a volume of the urine 50 in the canister 114. In some embodiments, the urine volume indicator 235 includes a float member 237 that floats on the urine 50 in the canister 114 and moves up and down along a string 234, wire, cable, or other member positioned in the interior region of the canister 114. The string 234 may extend from the lid 119 to a connector 232 secured or securable to the bottom region of the canister 114. The urine volume indicator 235 may include a gauge 239 that indicates the amount of the urine in the canister 114. For example, the gauge 239 may include numbers that display a volume or percent capacity of the canister 114 or colors that indicate relative capacity of the canister (e.g., green, yellow, and red, described above). The gauge 239 also may include a needle that moves responsive to movement of the float member 237 to one or more of the numbers or colors on the gauge to indicate the relative amount of the urine 50 in the canister 114. In some embodiments, a string, wire, or other member may extend between the float member 237 and the gauge 239 such that the needle on the gauge 239 moves responsive to up and down movement of the float member 237 in the canister 114. The urine volume indicator 235 may include one or more support members 236a, 236b, 236c, 236d configured to mount the urine volume indicator 235 to the pump 116 and/or the canister 114. The urine collection system 200 may include the urine alert device 155 configured to communicate with the urine volume indicator 235 as described above in relation to the urine volume indicator 135.

Turning now to FIG. 2B, a urine collection system 280 may include a urine volume indicator 285, according to an embodiment. Unless otherwise noted, the urine collection system 280 can include any aspect of the urine collection system 100. For example, the urine collection system 200 may include one or more (e.g., all) of the pump 116, the canister 114, the lid 119, the conduits 117a, 117b (not shown), the urine collection device 112 (not shown), and/or the urine alert device 155.

The urine volume indicator 285 may be secured or securable to at least one of the lid 119 or the canister 114 and configured to detect a property relating at least to a volume of the urine 50 in the canister 114. In some embodiments, the urine volume indicator 285 includes a float member 287 that floats on the urine 50 in the canister 114. A base 281 may be detachably or fixedly secured or securable to the lid 119 (shown in FIG. 2B) or a sidewall of the canister 114 (not shown), and a bracket or body 282 may extend from the base 218 into the interior region of the canister 114. A wheel 283 or dial forming at least a semi-circle may be pivotably secured to the body 282 to pivot about an axis, and an arm 284 connecting the float member 287 and the wheel 283. As the float member 287 rises with the urine in the canister 114, the portions of the wheel 283 may be exposed to indicate a numeric value of percent capacity and/or a color scale (green, yellow, and red, as described above) of percent capacity of the urine in the canister 114. In some embodiments, the urine collection system 280 may include a proximity sensor secured or securable to at least one of the lid 119 or the canister 114. The proximity sensor may include any sensor configured to detect the proximity of the float member 287 to the sensor, such as a Reed switch or a laser. The proximity sensor may generate a signal, such as an electrical signal, when the float is within a predetermined distance of the proximity sensor. The urine collection system 200 may include the urine alert device 155 configured to communicate with the urine volume indicator 235 and/or the proximity sensor as described above in relation to the urine volume indicator 135.

Turning now to FIG. 2C, a urine collection system 260 may include a base 267 having a urine volume indicator 265 secured thereto, according to an embodiment. Unless otherwise noted, the urine collection system 260 may include any aspect of other urine collection systems described herein. Although not shown or visible in FIG. 2C, the urine collection system may include any canister, pump, lid, conduit, urine collection device, pressure indicator, and/or flow indicator described herein. Aspects of the urine collection system 260 may be included in other urine collection systems described herein. The pump 116 may be housed in or secured to the base 267 and may be configured to draw air from the interior region of the canister 114.

In some embodiments, the base 267 includes a recess 262 sized to receive at least a portion of the canister 114. For example, at least a portion of the bottom region of the canister 114 may fit within the recess 262 and shaped complementary to the recess 262. The urine volume indicator 265 may include a scale positioned at least partially within the recess 262. The scale may include any scale, gauge, or sensor configured to weigh the canister 114. In some embodiments, the urine volume indicator 265 is positioned the bottom of the recess 262. The volume of the urine in the canister 114 may be determined by a change in weight of the canister 114 before, during, and/or after urine is collected from the user. In some embodiments, the urine volume indicator 265 may be configured to weigh the canister 114 at predetermined intervals, continuously upon activation, and/or continuously for a period of time responsive to detecting a change in weight. The urine volume indicator 265 may be configured to determine the volume of the urine in the canister 114 based on the weight measured by the scale.

In some embodiments, the urine volume indicator 265 includes an ultrasound transducer configured to detect the property relating at least to the volume of the urine in the canister 114. For example, the volume of the urine in the canister 114 may be determined using echo or time of flight of ultrasound waves through the canister 114. In some embodiments, the urine volume indicator 265 is positioned the bottom of the recess 262. In some embodiments, the ultrasound transducer may be positioned or embedded on the canister 114, the lid 119, or on a wall-mounted canister support for a wall-mounted pump or vacuum source.

The urine volume indicator 265 may be configured to transmit data relating to the volume of the urine in the canister 114 as measured by the urine volume indicator 265 to a controller, and the controller may be configured to determine the volume of the urine in the canister 114 based on the data measured by the urine volume indicator 265. In some embodiments, at least one of the urine volume indicator 265 or the controller may be configured to communicate with the alert device 155. For example, at least one of the urine volume indicator 265 or the controller may be configured to communicate with the alert device when the volume of the urine in the canister 114 is at a predetermined level or volume.

Turning now to FIG. 2D, a urine collection system 270 may include a base 277 that has a urine volume indicator secured thereto, according to an embodiment. Unless otherwise noted, the urine collection system 270 may include any aspect of other urine collection systems described herein. Although not shown or visible in FIG. 2D, the urine collection system may include any pump, conduits, urine collection device, pressure indicator, and/or flow indicator described herein. The pump 116 may be housed in or secured to the base 277 and may be configured to draw air from the interior region of the canister 114.

In some embodiments, the base 277 includes the recess 262 sized to receive at least a portion of the canister 114. For example, at least a portion of the bottom region of the canister 114 may fit within the recess 262 and be complementary to the recess 262. The urine volume indicator may include a scale 272 positioned and/or secured to the base 277. For example, the scale 272 may include one or more (e.g., two, three, four, five, etc.) feet secured to the bottom of the base 277. The scale 272 may include any scale, gauge, or sensor configured to weigh the canister 114 with the base 277. The volume of the urine in the canister 114 may be determined by a change in weight of the canister 114 and the base 277 during or after urine is collected from the user. In some embodiments, the scale 272 may be configured to weigh the canister 114 at predetermined intervals, continuously upon activation, and/or continuously for a period of time responsive to detecting a change in weight. The scale 272 may be configured to determine the volume of the urine in the canister 114 based on the weight measured by the scale 272. In some embodiments, the scale 272 may be configured to transmit data relating to the weight measured by the scale 272 to a controller, and the controller may be configured to determine the volume of the urine in the canister 114 based on the weight measured by the scale 272. In some embodiments, at least one of the scale 272 or the controller may be configured to communicate with the alert device 155. For example, at least one of the scale 272 or the controller may be configured to communicate with the alert device when the volume of the urine in the canister 114 is at a predetermined level or volume.

Turning now to FIG. 2E, a urine collection system 290 having a urine volume indicator may be used in a hospital or other care setting, according to an embodiment. Unless otherwise noted, the urine collection system 290 may include any aspect of other urine collection systems described herein. Moreover, aspects of the urine collection system 290 may be applied to any other urine collection system described herein, such as aspects of use in a hospital or care setting. In some settings, the urine collection system 290 may include a wall-mounted pump 296 or vacuum source in fluid communication with the second conduit 117b effective create a negative pressure within the canister 294. Aspects of the urine collection system 290, such as the urine volume indicator may be used in settings outside of hospital or care facilities, including home and portable use. As noted above, the urine collection device 112 may be positioned at least proximate to a urethra of the user 60, shown in FIG. 2E.

In some embodiments, the urine volume indicator may include a scale 298 mounted or mountable to a bedframe and configured to support at least the canister 294. In some embodiments, the scale 298 is mounted or mountable to the bedframe and configured to support the canister 114 and the base 107 (not shown), and the wall-mounted vacuum source 296 is absent. In some embodiments, the scale 298 may be mounted or mountable to other objects, such as a wheelchair, a night stand, or a wall. For example, the scale 298 may be mounted on the wall proximate to the wall-mounted vacuum source 296. The scale 298 may include any scale, gauge, or sensor configured to weigh at least the canister 294. The volume of the urine in the canister 294 may be determined by a change in weight of the canister 294 during or after urine is collected from the user 60. In some embodiments, the scale 298 may be configured to weigh the canister 294 at predetermined intervals, continuously upon activation, and/or continuously for a period of time responsive to detecting a change in weight. The scale 298 may be configured to determine the volume of the urine in the canister 294 based on the weight measured by the scale 298.

In some embodiments, the scale 298 may be configured to transmit data relating to the weight measured by the scale 298 to a controller. In some embodiments, the controller includes an external electronic device 295, such as an electronic device having a display monitor that is configured to determine and/or display the volume of urine in the canister 294. In some embodiments, the controller may include a controller housed in the base 107 supporting the canister 294. In some embodiments, at least one of the scale 298 or the controller may be configured to communicate with the alert device on the electronic device 295, in the room, at an aid station, or other electronic device. For example, at least one of the scale 298 or the controller may be configured to communicate with an alert device when the volume of the urine in the canister 294 is at a predetermined level or volume.

The urine volume indicator of the urine collection system 290 may include a sensor 292 secured to or embedded in at least one the canister 294 or the lid and configured to detect the property relating at least to the volume of the urine in the canister 294. The urine collection system 290 may include the sensor 292 in addition to the scale 298 or in alternative to the scale 298 (. e.g., the scale is absent). In some embodiments, the urine collection system 290 includes the scale 298, and the sensor 292 is absent (e.g., the urine collection system 290 includes the canister 114). The sensor 292 may include at least one of a conductive, impedance, or radio-frequency identification (RFID) sensor that is configured to detect the property relating at least to the volume of the urine in the canister 294. The sensor 292 also may be configured to wirelessly communicate with the external electronic device 295. The external electronic device 295 may be configured to use the data received from the sensor 292 to determine the volume of the urine in the canister 294. In some embodiments, the external electronic device may be configured to produce an alert signal when the volume of the urine in the canister 294 is at or surpassing a predetermined volume. In some embodiments, the external electronic device 295 may be configured to transmit an alert message to one or more other electronic devices indicating that the volume of the urine in the canister is at or surpassing the predetermined volume.

Returning now to FIG. 1B, the urine collection system 100 may include the flow indicator 125 to allow a user or caregiver to confirm that air or fluid is flowing in the urine collection system 100. The flow indicator 125 may be positioned between the canister 114 and the urine collection device 112 and configured to detect a property relating at least to a flow of fluid through the first conduit 117a. In some embodiments, the flow indicator 125 may be positioned in-line with the first conduit 117a to indicate fluid and/or air are flowing through the first conduit 117a. The flow indicator 125 may be positioned in-line between the canister 114 and the urine collection device 112, and may be configured to detect a property relating at least to a flow of fluid through the first conduit 117a. In some embodiments, an additional flow indicator may be similarly secured or positioned on the second conduit 117b to detect a property relating at least to a flow of air through the second conduit 117b.

The flow indicator 125 may utilize the motion of air or fluid in the first conduit 117a to provide a visual or audible indication of the motion or flow of air or fluid in the first conduit 117a. In some embodiments, the flow indicator 125 includes an enclosed wheel that spins or rotates responsive to fluid flow through the indicator 125. Thus, a user or caregiver can quickly view the flow indicator and determine if air or liquid is flowing through the first conduit 117a by whether or not the enclosed wheel is spinning or rotating. If the pump 116 is activated and the enclosed wheel of the flow indicator is not spinning or rotating, at least one of the first conduit 117a or the second conduit 117b may be kinked (e.g., have a sharp twist, bend, or curve that inhibits or prevents the flow of fluid through the respective conduit), and/or the canister 114 may not be sealed. When one of the first conduit 117a or the second conduit 117b are kinked and/or the canister 114 is not sealed, urine voided into the urine collection device 112 may not be drawn into the canister 114, and may instead overflow from the urine collection device 112. Thus, the flow indicator 125 in at least one, some, or all embodiments prevent undesired and unhygienic displacement of urine on or around the user.

Turning ahead in the drawings to FIG. 3, in some embodiments, a flow indicator 300 may replace the flow indicator 125. The flow indicator 300 may be configured to detect a property relating at least to a flow of fluid through the first conduit 117a. In some embodiments, the flow indicator 300 includes a tube 302 and a float 310 or ball movable in the tube 302 responsive to fluid flow through the flow indicator 300. For example, the float 310 may be at a first position in the tube 302 when no fluid flow is present. The float 310 may raise or lower within the tube 302 when fluid flow is present. In some embodiments, the tube 305 includes one or more markers 305 positioned on the tube 302 to indicate a predetermined fluid flow is present in the first conduit 117a when the float 310 reaches the one or more markers 305. In some embodiments, the one or more markers 305 each indicate a flow rate of fluid through the flow indicator 300. The flow indicator 300 also may include a gauge 306 having a needle and a numeric and/or colored scale indicating a rate or relative rate of flow fluid through the flow indicator 300. For example, the needle in the gauge 306 may move responsive to fluid flow to a number indicating the approximate rate of fluid flow through the flow indicator 300. In some embodiments, the needle in the gauge 306 may move to a color (e.g., red, yellow, or green) indicating an acceptable or unacceptable rate of fluid flow through the fluid indicator 300.

In some embodiments of the fluid collection system 100, fluid indicator 125, 300 may be configured to communicate with the alert device 155, and the alert device 155 may be configured to produce an alert signal when the pump 116 is activated but the flow indicator 125, 300 indicates that no fluid flow is present or the fluid flow is below a predetermined level. The alert signal produced responsive to the flow indicator 125, 300 may include at least one of a visual (e.g., light) alert, an audible (e.g., sound) alert, or a haptic alert. In some embodiments, the visual alert or the audible alert associated with the flow indicator 125, 300 may be different than the visual alert or the audible alert produced responsive to the urine volume indicator 135. The flow indicator 125, 300 may be wired or wirelessly coupled to the pump 116 in the urine collection system 100 effective to deactivate (e.g., turn off) the pump when the pump 116 is activated and the flow indicator 125, 300 indicates no fluid flow is present in the first conduit 117a. The urine collection system 100 also may include a controller configured to facilitate communication between the flow indicator 125, 300 and at least one (e.g., both) of the pump 116 and the alert device 155.

Returning to FIG. 1B, the urine collection system 100 may include a pressure indicator 115 secured to or positioned on the canister 114 or the lid 119 and configured to detect a property relating at least to a pressure in the interior region of the canister 114. During use of the urine collection system 100, a user or caregiver may desire to confirm that there is pressure in the canister 114 of the urine collection system 100. If the desired negative pressure is absent from the canister 114 while the pump 116 is activated, urine may not be drawn from the urine collection device 112 as desired, resulting in urine overflowing from the urine collection device 112 as urine is voided by the user. In some embodiments, the pressure sensor 115 may include an electric pressure sensor positioned within the canister 114, such as secured to an interior surface of the canister 114 or the lid 119.

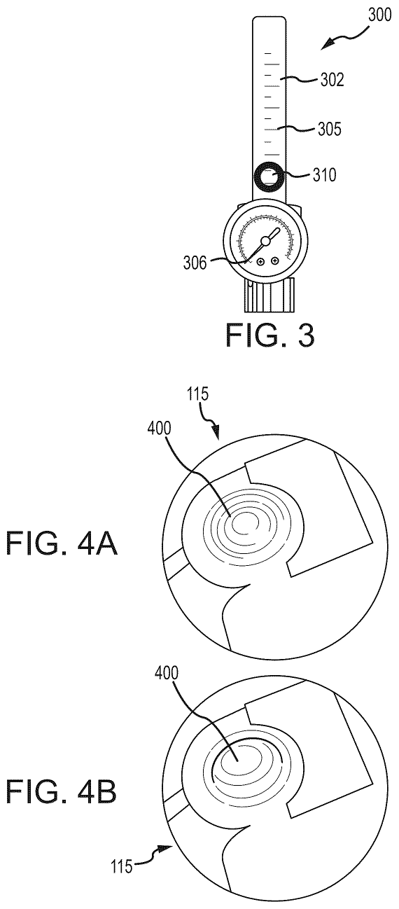

Turning ahead in the drawings to FIGS. 4A and 4B, in some embodiments, the pressure indicator 115 includes a button 400 positioned on the lid 119 or the canister 114. The button 400 may be configured to extend outward (shown in FIG. 4B) when a negative pressure is absent in the interior region of the canister 114, e.g., the pump 116 is not activated. The button 400 also may be configured to extend inward (shown in FIG. 4A) when the negative pressure is present in the interior region of the canister 114, e.g., the pump 116 is activated. A user or caregiver, then, can view the pressure indicator 115 and quickly determine if the desired negative pressure is present in the interior region of the canister 114 while the pump 116 is activated. If, for example, the pump 116 is activate but the button 400 remains extended outward, the second conduit 117b may be kinked or the canister 114 may not be properly sealed.

In some embodiments of the fluid collection system 100, pressure indicator 115 may be configured to communicate with alert device 155, and the alert device 155 may be configured to produce an alert signal when the pump 116 is activated but the pressure indicator 115 indicates that a negative pressure is absent from the interior region of the canister 114. The alert signal produced responsive to the pressure indicator 115 may include at least one of a visual (e.g., light) alert, an audible (e.g., sound) alert, or a haptic alert. In some embodiments, the visual alert or the audible alert associated with the pressure indicator 115 may be different than the visual alert or the audible alert produced responsive to the urine volume indicator 135 and/or the flow indicator 125. The pressure indicator 115 may be wired or wirelessly coupled to the pump 116 in the urine collection system 100 effective to activate (e.g., turn off) the pump when the pump 116 is activated and the pressure indicator 115 indicates that a negative pressure is absent from the interior region of the canister 114. The urine collection system 100 also may include a controller configured to facilitate communication between the pressure indicator 115 and at least one (e.g., both) of the pump 116 and the alert device 155.

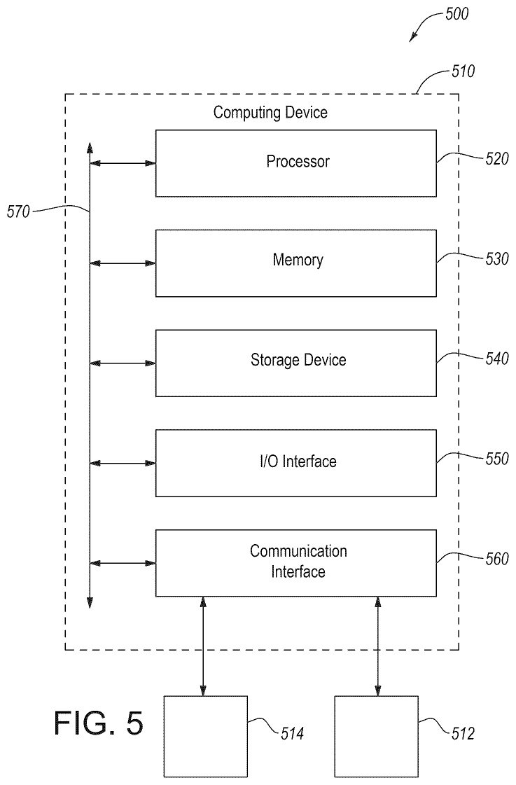

As noted above, the urine collection systems described herein may include a controller that may be configured to communicate with one or more (e.g., all) of the alert device 155, the pressure indicator 115, the flow indicator 125, 300, and/or the urine volume indicator 135, 235, 265, 272, 285, 292, 298. FIG. 5 is a schematic of a controller 500 that may be included in any of the urine collection systems described herein. For example, the controller may be housed in or secured to the base of the urine collection system, and/or may be included in one or more (e.g., all) of the alert device 155, the pressure indicator 115, the flow indicator 125, 300, and/or the urine volume indicator 135, 235, 265, 272, 285, 292, 298.

The controller 500 includes at least one computing device 510. The at least one computing device 510 is an exemplary computing device that may be configured to perform one or more of the acts described herein. The at least one computing device 510 can include one or more servers, one or more computers (e.g., desk-top computer, lap-top computer), or one or more mobile computing devices (e.g., smartphone, tablet, etc.). The computing device 510 can comprise at least one processor 520, memory 530, a storage device 540, an input/output (“I/O”) device/interface 550, and a communication interface 560. While an example computing device 510 is shown in FIG. 5, the components illustrated in FIG. 5 are not intended to be limiting of the controller 500 or computing device 510. Additional or alternative components may be used in some examples. Further, in some examples, the controller 500 or the computing device 510 can include fewer components than those shown in FIG. 5. For example, the controller 500 may not include the one or more additional computing devices 512, 514. In some examples, the at least one computing device 510 may include a plurality of computing devices, such as a server farm, computational network, or cluster of computing devices. Components of computing device 510 shown in FIG. 5 are described in additional detail below.

In some examples, the processor(s) 520 includes hardware for executing instructions (e.g., instructions for carrying out one or more portions of any of the methods disclosed herein), such as those making up a computer program. For example, to execute instructions, the processor(s) 520 may retrieve (or fetch) the instructions from an internal register, an internal cache, the memory 530, or a storage device 540 and decode and execute them. As an example, the processor(s) 520 may include one or more instruction caches, one or more data caches, and one or more translation lookaside buffers (TLBs). Instructions in the instruction caches may be copies of instructions in memory 530 or storage device 540. In some examples, the processor 520 may be configured (e.g., include programming stored thereon or executed thereby) to carry out one or more portions of any of the example methods disclosed herein.

In some examples, the processor 520 is configured to perform any of the acts described herein and/or cause one or more portions of the computing device 510 or controller 500 to perform at least one of the acts disclosed herein. Such configuration can include one or more operational programs (e.g., computer program products) that are executable by the at least one processor 520. For example, the processor 520 may be configured to automatically communicate with the alert device 155 and/or communicate with the pump to activate or deactivate the pump.

The at least one computing device 510 (e.g., a server) may include at least one memory storage medium (e.g., memory 530 and/or storage device 540). The computing device 510 may include memory 530, which is operably coupled to the processor(s) 520. The memory 530 may be used for storing data, metadata, and programs for execution by the processor(s) 520. The memory 530 may include one or more of volatile and non-volatile memories, such as Random Access Memory (RAM), Read Only Memory (ROM), a solid state disk (SSD), Flash, Phase Change Memory (PCM), or other types of data storage. The memory 530 may be internal or distributed memory.

The computing device 510 may include the storage device 540 having storage for storing data or instructions. The storage device 540 may be operably coupled to the at least one processor 520. In some examples, the storage device 540 can comprise a non-transitory memory storage medium, such as any of those described above. The storage device 540 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 540 may include removable or non-removable (or fixed) media. Storage device 540 may be internal or external to the computing device 510. In some examples, storage device 540 may include non-volatile, solid-state memory. In some examples, storage device 540 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 530 and/or storage device 540 (e.g., memory storage medium(s)) may store one or more databases thereon.

In some examples, one or more of urine collection history, pump history, or alert history may be stored in a memory storage medium such as one or more of the at least one processor 520 (e.g., internal cache of the processor), memory 530, or the storage device 540. In some examples, the at least one processor 520 may be configured to access (e.g., via bus 570) the memory storage medium(s) such as one or more of the memory 530 or the storage device 540. For example, the at least one processor 520 may receive and store the data (e.g., look-up tables) as a plurality of data points in the memory storage medium(s). The at least one processor 520 may execute programming stored therein adapted access the data in the memory storage medium(s).

The computing device 510 also includes one or more I/O devices/interfaces 550, which are provided to allow a user to provide input to, receive output from, and otherwise transfer data to and from the computing device 510. These I/O devices/interfaces 550 may include a mouse, keypad or a keyboard, a touch screen, camera, optical scanner, network interface, web-based access, modem, a port, other known I/O devices or a combination of such I/O devices/interfaces 550. The touch screen may be activated with a stylus or a finger.

The I/O devices/interfaces 550 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 550 are configured to provide graphical data to a display for presentation to a user. The graphical data may be representative of one or more graphical user interfaces and/or any other graphical content as may serve a particular implementation.

The computing device 510 can further include a communication interface 560. The communication interface 560 can include hardware, software, or both. The communication interface 560 can provide one or more interfaces for communication (such as, for example, packet-based communication) between the computing device 510 and one or more additional computing devices 512, 514 or one or more networks. The one or more additional computing devices, for example, may include any of the indicators described herein or external electronic devices. The one or more additional computing devices may include any aspect of the controller 500 described herein. For example, communication interface 560 may include a network interface controller (NIC) or network adapter for communicating with an Ethernet or other wire-based network or a wireless NIC (WNIC) or wireless adapter for communicating with a wireless network, such as a WI-FI.

Any suitable network and any suitable communication interface 560 may be used. For example, computing device 510 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 500 or computing device 510 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. Computing device 510 may include any suitable communication interface 560 for any of these networks, where appropriate.

The computing device 510 may include a bus 570. The bus 570 can include hardware, software, or both that couples components of computing device 510 to each other. For example, bus 570 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

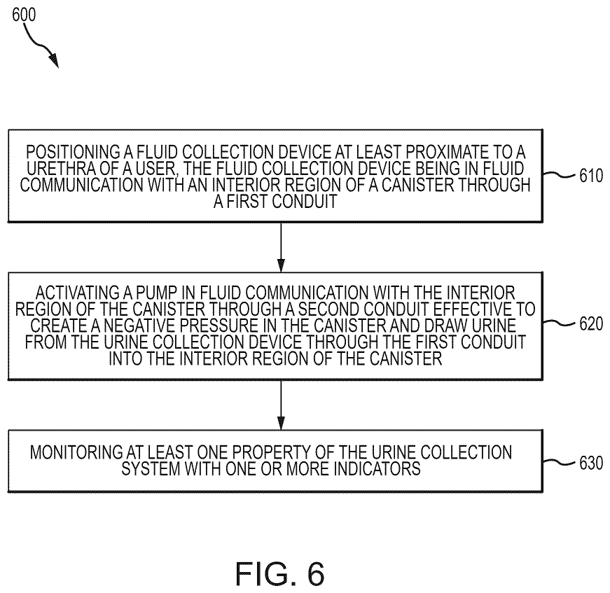

FIG. 6 is a flow diagram of a method 600 of collecting urine from a user, according to an embodiment. The method 600 includes an act 610 positioning a fluid collection device at least proximate to a urethra of a user. The fluid collection device may be in fluid communication with an interior region of a canister through a first conduit. The method 600 includes an act 620 of activating a pump in fluid communication with the interior region of the canister through a second conduit effective to create a negative pressure in the canister and draw urine from the urine collection device through the first conduit into the interior region of the canister. The method 600 includes an act 630 of monitoring at least one property of the urine collection system with one or more indicators.

In some embodiments, the act 630 includes monitoring at least a property relating at least to a volume of urine in the canister with a urine volume indicator secured to the canister. In these and other embodiments, the method 600 may include an act of deactivating the pump when the urine volume indicator indicates the volume of the urine in the canister has reached a predetermined volume.

In some embodiments, the act 630 includes monitoring at least a property relating at least to a flow of fluid through the first conduit with a flow indicator in fluid communication with the first conduit and positioned between the canister and the urine collection device. In these and other embodiments, the method 600 may include an act of removing a kink in the first conduit when the fluid indicator indicates the flow of the fluid through the first conduit is absent or below a predetermined rate after the pump has been activated.

In some embodiments, the act 630 includes monitoring at least a property relating at least to a pressure in the interior region of the canister with a pressure indicator secured to the canister. In these and other embodiments, the method 600 may include at least one act of sealing a lid on the canister or removing a kink in the second conduit when the pressure indicator indicates an absence of negative pressure in the interior region of the canister after the pump has been activated.

The acts of the method 600 described above are for illustrative purposes. For example, the acts of the method 600 can be performed in different orders, split into multiple acts, modified, supplemented, or combined. In an embodiment, one or more of the act of the method 600 can be omitted from the method 600. Any of the acts of the method 600 can include using any of the urine collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.” While various aspects and embodiments have been disclosed herein, other aspects and embodiments are contemplated. The various aspects and embodiments disclosed herein are for purposes of illustration and are not intended to be limiting.

1. A urine collection system, comprising:
a urine collection device configured to be positioned at least proximate to a urethra of a user;
a canister having a sidewall defining an interior region;
a first conduit in fluid communication with the urine collection device and the canister;
a urine volume indicator configured to detect a property relating at least to a volume of urine in the interior region of the canister;
a pump configured to draw urine from the urine collection device through the first conduit and into the interior region of the canister; and
a controller operably coupled to the pump and the urine volume indicator, the controller configured to control the pump based on the volume of the urine detected by the volume indicator, including automatically activating the pump when the volume detected is below a first predetermined volume and automatically deactivating the pump when the volume detected is at or above a second predetermined volume.

2. The urine collection system of claim 1, further comprising a lid secured or securable to the canister, wherein the urine volume indicator includes:
a float member configured to float on the urine in the canister;
a body secured to an inner surface of the lid or an inner surface of the sidewall of the canister;
a wheel or dial forming at least a semi-circle pivotably secured to the body to pivot about an axis and including colors representing percent capacity of the canister; and
an arm connecting the float member to the wheel, wherein the wheel or dial rotates responsive to movement of the float member to expose a color of the colors representing the percent capacity of the canister.

3. The urine collection system of claim 1, further comprising a base housing the pump and including a recess sized to receive at least a portion of the canister within the recess, wherein the urine volume indicator includes a scale positioned in the recess of the base below an upper rim of the base defining the recess.

4. The urine collection system of claim 1, further comprising a base housing the pump and including a recess sized to receive at least a portion of the canister within the recess, wherein the urine volume indicator includes an ultrasound transducer positioned in the recess of the base below an upper rim of the base.

5. The urine collection system of claim 1, wherein the urine volume indicator includes an ultrasound transducer positioned on at least one of the canister or a lid secured to the canister.

6. The urine collection system of claim 1, wherein the pump includes a vacuum source configured to produce a negative pressure effective to draw urine from the urine collection device through the first conduit and into the interior region of the canister.

7. The urine collection system of claim 1, further comprising:
a flow indicator operably coupled to the controller and positioned to detect a property relating at least to a flow of fluid through the first conduit between the canister and the urine collection device; and
an alert device operably coupled to the controller and configured to produce an alert signal when the pump is activated but the flow indicator indicates that no fluid flow is present through the first conduit or the fluid flow in the first conduit is below a predetermined level.

8. The urine collection system of claim 7, wherein the controller is configured to automatically deactivate the pump when the pump is activated and the flow indicator indicates that no fluid flow is present through the first conduit or the fluid flow in the first conduit is below a predetermined level.

9. A method of collecting urine from a user, comprising:
positioning a fluid collection device at least proximate to a urethra of a user, the fluid collection device being in fluid communication with an interior region of a canister through a first conduit;
automatically activating a pump in fluid communication with the interior region of the canister effective to create a negative pressure in the canister and draw urine from the urine collection device through the first conduit into the interior region of the canister when a urine volume indicator detects urine in the canister is below a predetermined volume; and
automatically deactivating the pump when the urine volume indicator detects the urine in the canister is at or above a predetermined volume.

10. The method of claim 9, wherein:
a lid is secured to the canister;
the urine volume indicator includes:
a float member configured to float on the urine in the canister;
a body secured to an inner surface of the lid or an inner surface of the sidewall of the canister;
a wheel or dial forming at least a semi-circle pivotably secured to the body to pivot about an axis and including colors representing percent capacity of the canister; and
an arm connecting the float member to the wheel, wherein the wheel or dial rotates responsive to movement of the float member to expose a color of the colors representing the percent capacity of the canister.

11. The method of claim 9, further comprising positioning at least a portion of the canister within a recess of a base housing the pump, wherein the urine volume indicator includes a scale positioned in the recess of the base below an upper rim of the base defining the recess.

12. The method of claim 9, further comprising positioning at least a portion of the canister within a recess of a base housing the pump, wherein the urine volume indicator includes an ultrasound transducer positioned in the recess of the base below an upper rim of the base.

13. The method of claim 9, wherein the urine volume indicator includes an ultrasound transducer positioned on at least one of the canister or a lid secured to the canister.

14. The method of claim 9, wherein automatically activating a pump includes automatically activating the pump of a vacuum source to create the negative pressure in the canister and draw the urine from the urine collection device through the first conduit into the interior region of the canister.

15. The method of claim 9, further comprising:
detecting a property relating at least to a flow of fluid through the first conduit between the canister and the urine collection device with a flow indicator; and
producing an alert with an alert device when the pump is activated but the flow indicator indicates that no fluid flow is present through the first conduit or the fluid flow in the first conduit is below a predetermined level.

16. The method of claim 15, further comprising automatically deactivating the pump when the pump is activated and the flow indicator indicates that no fluid flow is present through the first conduit or the fluid flow in the first conduit is below a predetermined level.

17. A method of collecting urine from a user, comprising:
positioning a fluid collection device at least proximate to a urethra of a user, the fluid collection device being in fluid communication with an interior region of a canister through a first conduit;
activating a pump in fluid communication with the interior region of the canister effective to create a negative pressure in the canister and draw urine from the urine collection device through the first conduit into the interior region of the canister;
detecting a property relating at least to a flow of fluid through the first conduit between the canister and the urine collection device with a flow indicator; and
producing an alert with an alert device when the pump is activated but the flow indicator indicates that no fluid flow is present through the first conduit or the fluid flow in the first conduit is below a predetermined level.

18. The method of claim 17, further comprising automatically deactivating the pump when the pump is activated and the flow indicator indicates that no fluid flow is present through the first conduit or the fluid flow in the first conduit is below a predetermined level.

19. The method of claim 17, further comprising:
automatically activating the pump when a urine volume indicator detects urine in the canister is below a predetermined volume; and
automatically deactivating the pump when the urine volume indicator detects the urine in the canister is at or above a predetermined volume.

20. The method of claim 19, wherein:
a lid is secured to the canister;
the urine volume indicator includes:
a float member configured to float on the urine in the canister;
a body secured to an inner surface of the lid or an inner surface of the sidewall of the canister;
a wheel or dial forming at least a semi-circle pivotably secured to the body to pivot about an axis and including colors representing percent capacity of the canister; and
an arm connecting the float member to the wheel, wherein the wheel or dial rotates responsive to movement of the float member to expose a color of the colors representing the percent capacity of the canister.
